(19) United States
(12) Patent Application Publication
(10) Pub. No.: US 2010/0137248 A1
(43) Pub. Date: Jun. 3, 2010

(54) BORON-CONTAINING COMPOUNDS, USES AND PREPARATION THEREOF

(75) Inventor: André Vachereau, Roxboro (CA)

Correspondence Address: BERESKIN AND PARR LLP/S.E.N.C.R.L., s.r.l., 40 KING STREET WEST, BOX 401, TORONTO, ON, M5H 3Y2, CA

(73) Assignee: DINOTEC INC., Roxboro, QC (CA)

(21) Appl. No.: 12/596,712

(22) PCT Filed: Apr. 18, 2008
(86) PCT No.: PCT/CA2008/000735

§ 371 Date: Oct. 20, 2009

Related U.S. Application Data
(60) Provisional application No. 60/913,013, filed on Apr. 20, 2007.
(60) Provisional application No. 60/981,927, filed on Oct. 23, 2007.

(51) Int. Cl. A61K 31/69; C07F 5/04; A61P 19/00; A61P 31/04; A61P 29/00; A61P 17/02

(52) U.S. Cl. 514/64; 558/290; 558/291

(57) ABSTRACT

There are provided compounds of formulas (I) to (XIV) and compositions comprising such compounds. Such compounds and compositions are particularly useful for treating inflammation and/or pain, or for water treatment.

### FIELD OF THE INVENTION

The present invention relates to the field of chemistry. In particular, it relates to boron-containing compounds. These compounds can be used for various purposes such as anti-inflammatory agents and/or analgesic agents for the treatment of musculo-skeletal disorders and other conditions involving pain. They can also be used for water treatment.

### BACKGROUND OF THE INVENTION

Musculo-skeletal diagnoses include the two most common forms of arthritis, osteoarthritis and rheumatoid arthritis, muscular low back pain and muscular neck pain and fibromyalgia. Injuries affecting the musculo-skeletal system which includes the bones, joints, muscles, tendons and ligaments are also included. For patients with “arthritis/rheumatism” and related conditions, the use of medications commonly prescribed for these conditions are grouped as following: (1) Non-steroidal anti-inflammatory drugs (NSAIDs), (2) Corticosteroids, (3) Disease-modifying anti-rheumatic drugs (DMARDs) and (4) Biologic response modifiers (biologics).

(1) Non-Steroidal Anti-Inflammatory Drugs (NSAIDs)

There are two classes of NSAIDs: (a) conventional or non-selective ones, such as acetylsalicylic acid (Aspirin®), acetaminophen (Tylenol®, Tempra®), ibuprofen (Motrin®, Advil®), ketoprofen (Orudis®), naproxen (Naprosyn®, Aleve®), and diclofenac (Voltaren®). (b) The COX-2 inhibitor or selective ones, such as rofecoxib (Vioxx®), celecoxib (Celebrex®) and valdecoxib (Bextra®).

Conventional NSAIDs drugs are widely used by a variety of patients that can access the products either by prescriptions (examples: naproxen, diclofenac) or as OTC drugs (examples: acetylsalicylic acid, acetaminophen, ibuprofen). Although their effectiveness has been proved in acute pain syndromes that are inflammatory, their efficacy in controlling chronic pain is not well established. Also all these NSAIDs reach a limit to their maximum analgesics effect, even taken at “grams level”. The major drawback in the utilization of traditional NSAIDs are associated with serious drug adverse effects that very often they are worse that the illness for which the drug was prescribed. The major risk with such NSAIDs is related to a specific gastrointestinal (GI) adverse effect that causes GI bleeding and causes an estimated 16,500 deaths each year in the United States alone. Also ibuprofen, naproxen and ketoprofen were considered by FDA (last February 2005) as product that could increase risk of CV events and asked all manufacturers of such products to change label to better inform consumers regarding the safe use of these products. Although acetaminophen (Tylenol®, Tempra®) do not cause the characteristic GI side effects of NSAIDs, an overdosage can cause serious and fatal hepatic complications even in patients with no underlying liver problems. COX-2 inhibitor drugs are recent NSAIDs products that selectively block the cyclooxygenase isoform 2 (COX-2) but not the isoform 1 (COX-1), associated with the GI safety problems encountered by most conventional NSAIDs drugs. Therefore COX-2 inhibitor medications were expected as effective as other NSAIDs but without known side effects. However these breakthrough medicines firstly released on market in 1999 (i.e.: rofecoxib or Vioxx® followed by celeroxib and valdecoxib for Celebrex® and Bextra®) were mostly removed from market in 2005 (Vioxx® and Bextra®) because of severe cardiovascular (CV) adverse events that lead to the deaths of several patients (i.e.: currently still in investigation by the FDA). It seems that the CV risk associated with COX-2 inhibitors represent a “class effect” that includes all similar drugs. As a result, FDA also required that the remaining COX-2 inhibitor product on market, celecoxib (Celebrex®) be considered as an high risk product for chronically ill patients with arthritis for example, that don't have any other treatment alternative, because of GI bleeding problems with other NSAIDs. Of course, as long as they do not have any other underlying CV problems.

(2) Corticosteroids

These agents can reduce temporarily pain and inflammation in patients afflicted with various rheumatic diseases. However corticosteroids are rarely used for long term treatment (i.e.: either oral or injection routes) due to the well-known side-effect profile of these medications that adversely hit badly several systems such as cardiovascular and immunity. Even when corticosteroids are delivered by injection in an affected joint for example (i.e. with expected few adverse effects), the number of injections per joint is limited to four on an annual basis. Therefore corticosteroids are definitely not a first line therapy even in severe pain condition associated with inflammation.

(3) Disease-Modifying Anti-Rheumatic Drugs (DMARDs)

These products are recommended as first line therapy for patients with rheumatoid arthritic that failed to respond to other therapies. DMARDs work by slowing down inflammation and associated pain but these products are responsible to cause severe side-effects that concern treating physicians when they balance risk/benefit of such medications. Typical examples of DMARDs are methotrexate, sulfasalazine, auranofin, gold sodium thiomalate and aurothioglucose, both used by injection.

(4) Biologic Response Modifiers (Biologic)

This is a new class of drugs for treating inflammatory conditions such as rheumatoid arthritis and for preventing disease progression. Biologic work much more quickly than DMARDs. However most of those products are injectable that are responsible to cause severe adverse effects (serious infections, liver damage, birth defects, bone marrow dysplasia, etc) including fatalities. Annual drug cost is also an issue and is estimated between 12,500$US to 18,000$US per patient. Four biologics are currently available on market: etanercept (Enbrel®), infliximab (Remicade®), anakinra (Kineret®) and leflonomide (Arava®).

Therefore even if the number of patients with musculo-skeletal problems such as arthritis is on the rise (i.e. 60 millions Americans estimated to be affected with the chronic form of arthritis, by 2020, according to an FDA report), paradoxically, the number of safe drug options to control inflammation associated with pain (i.e.: plus other musculo-skeletal disorders with pain primarily) is declining to almost nothing. Inflammation can be defined as a local response to cellular injury that initiates the elimination of noxious agents and damaged tissue. It is thus a complex process of physiological and immunological events characterized by capillary dilation, leukocyte infiltration, redness, heat, pain and swelling. Polymorphonuclear neutrophils (hereinafter, “PMNs”) and eosinophils are among the list of important mediators of inflammation and release growth factors, cytokines, prostaglandins, leukotrienes and proteases that exacerbate tissue damage. PMN-derived serine proteases such as elastase and cathepsin G are known pathogenic factors in inflammatory and degenerative diseases that include abnormal tissue catabolism. Although the inflammatory response can be regulated by anti-inflammatory agents such as corticocosteroids, immunosuppressants, non steroidal anti-inflammatory drugs (NSAIDs), COX-2 inhibitors and protease inhibitors, many of these agents have significant side effects. Corticosteroids may induce Cushingoid features, skin thinning, increased susceptibility to infection and suppression of the hypothalamic-pituitary-adrenal axis. Immunosuppressants may induce hypertension and nephrotoxicity.

U.S. Pat. No. 6,696,419 describes compositions and methods for treating inflammation. In particular, it relates to a formulation including a boron containing complex having a tetrahedral boron atom, which is bound to four ligands. The formulation is used to reduce inflammation when administered.

U.S. Patent Application published under No. 20050112118 a method for treating inflammation and inflammatory disorders. The method comprises the step of administering to a patient in need of such treatment a compound capable of interfering with the interaction between a first protein which is PRAK and a second protein which is ERK3.

However, all the previously proposed solutions presented at least some drawbacks. Some of them encompassed some serious secondary effects and some others resulted in relatively costly products. Thus, there is still a need to provide new compounds and compositions for use as an anti-inflammatory agent.

### SUMMARY OF THE INVENTION

In accordance with one aspect of the present invention there are provided compounds of formulas (I), (II), (III), (IV), (V), (VI), (VII), (VIII), (IX), (X), (XI), (XII), (XIII), and (XIV):

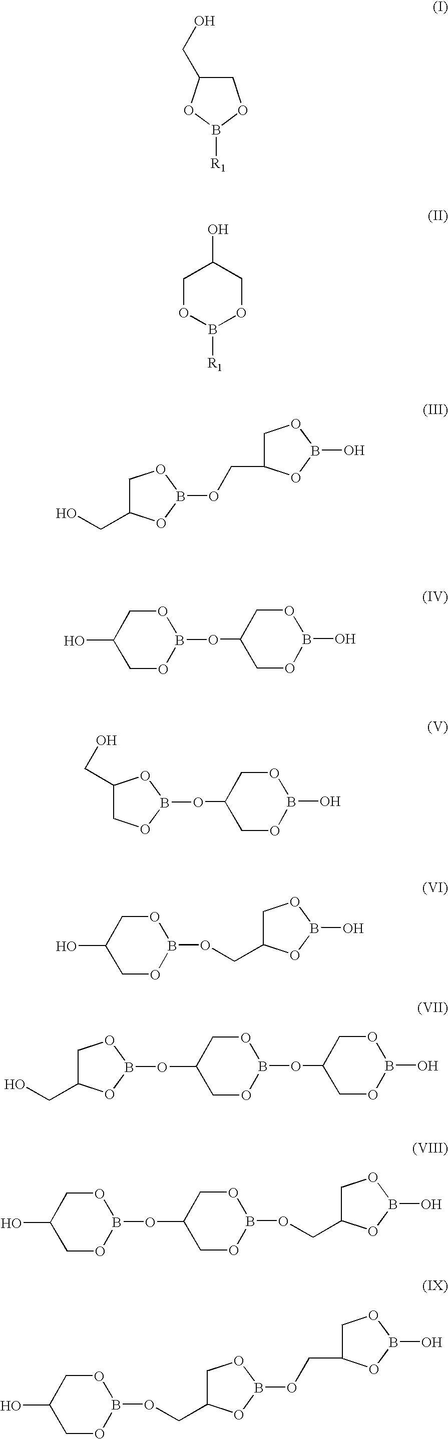

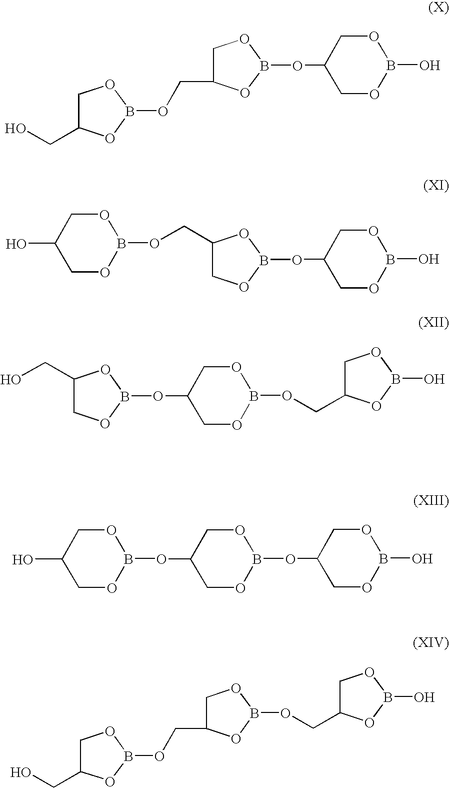

wherein
R₁ is —OH, a C₁-C₁₂ alkyl, a C₂-C₁₂ alkenyl, a C₂-C₁₂ alkynyl, a C₃-C₈ cycloalkyl, a C₃-C₈ cycloalkenyl, a C₆-C₁₂ aryl, or a C₁-C₁₂ heteroaryl group,
the alkyl, alkenyl, alkynyl, cycloalkyl, cycloalkenyl, aryl, and heteroaryl groups being unsubstituted or substituted by a hydroxy, a C₁-C₁₂ alkyl, a C₁-C₆ hydroxyalkyl, or a C₁-C₆ alkoxy.

It has been found that the compounds of the present invention are very efficient for treating inflammation. They thus also represent an advantageous alternative to the compounds of the prior art. In fact, they have been found to be very effective to reduce inflammation, preferably when administered topically on the site of inflammation. These compounds can also be easily prepared at low costs and their synthesis is simple and expedient. Some of these compounds can be rapidly prepared in a single step from commercially available intermediates.

In accordance with another aspect of the present invention, there is provided a composition comprising at least two compounds as previously defined.

In accordance with another aspect of the present invention there is provided a composition comprising at least one compound as previously defined, and a pharmaceutically acceptable carrier.

In accordance with another aspect of the present invention there is provided a composition comprising at least one compound chosen from compounds of formulas (I) and (II), and at least one compound chosen from compounds of formulas (III), (IV), (V), (VI), (VII), (VIII), (IX), (X), (XI), (XII), (XIII), and (XIV).

In accordance with another aspect of the present invention there is provided a composition comprising at least one compound chosen from compounds of formulas (XVI) and (XVII):

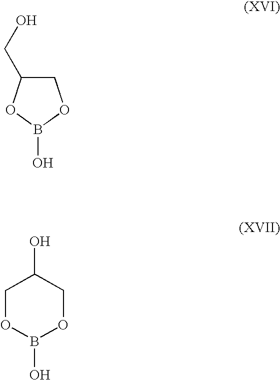

and at least one oligomer thereof.

In accordance with another aspect of the present invention there is provided a kit for treating inflammation and/or pain comprising at least one compound as previously defined, and instructions for using the same.

In accordance with another aspect of the present invention there is provided a kit for treating inflammation and/or pain including a composition comprising at least one compound as previously defined and a pharmaceutically acceptable carrier, and instructions for using the same.

In accordance with another aspect of the present invention there is provided a kit for treating musculo-skeletal disorders including a composition comprising at least one compound as previously defined and a pharmaceutically acceptable carrier, and instructions for using the same.

In accordance with another aspect of the present invention there is provided a method for treating inflammation and/or pain in a subject in need thereof, comprising administering to the subject an effective amount of at least one compound as previously defined.

In accordance with another aspect of the present invention there is provided a method for treating musculo-skeletal disorders in a subject in need thereof, comprising administering to the subject an effective amount of at least one compound as previously defined.

In accordance with another aspect of the present invention there is provided a method for treating inflammation and/or pain in a subject in need thereof, comprising administering to the subject an effective amount of at least one composition as previously defined.

In accordance with another aspect of the present invention there is provided a method for treating musculo-skeletal disorders in a subject in need thereof, comprising administering to the subject an effective amount of at least one composition as previously defined.

In accordance with another aspect of the present invention there is provided a method for treating wounds and/or burns in a subject in need thereof, comprising administering to the subject an effective amount of at least one compound as previously defined.

In accordance with another aspect of the present invention there is provided a method for treating wounds and/or burns in a subject in need thereof, comprising administering to the subject an effective amount of at least one composition as previously defined.

In accordance with another aspect of the present invention there is provided the use of at least one compound or composition as previously defined for treating inflammation and/or pain in a subject in need thereof.

In accordance with another aspect of the present invention there is provided the use of at least one compound or composition as previously defined for treating musculo-skeletal disorders in a subject in need thereof.

In accordance with another aspect of the present invention there is provided the use of at least one compound or composition as previously defined for wound healing and/or burns treatment.

In accordance with another aspect of the present invention there is provided the use of at least one compound or composition of the present invention for water treatment.

In accordance with another aspect of the present invention there is provided a method for treating water. The method comprises contacting the water to be treated with at least one compound or at least one composition of the present invention.

In accordance with another aspect of the present invention there is provided a process for preparing a compound of formula (I) and/or (II):

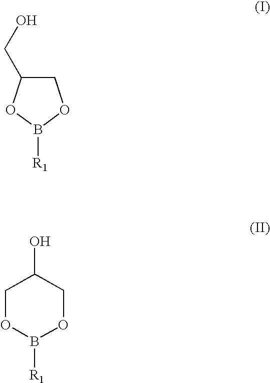

wherein
R₁ is —OH, a C₁-C₁₂ alkyl, a C₂-C₁₂ alkenyl, a C₂-C₁₂ alkynyl, a C₃-C₈ cycloalkyl, a C₃-C₈ cycloalkenyl, a C₆-C₁₂ aryl, or a C₁-C₁₂ heteroaryl group,
the alkyl, alkenyl, alkynyl, cycloalkyl, cycloalkenyl, aryl, and heteroaryl groups being unsubstituted or substituted by a hydroxy, a C₁₂ alkyl, a C₁-C₆ hydroxyalkyl, or a C₁-C₆ alkoxy,
   the process comprising reacting a compound of formula (XV):

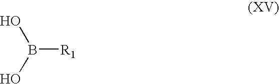

wherein R₁ is as previously defined,

with glycerol so as to obtain the compound of formula (I) and/or the compound of formula (II).

The term “alkyl” as used herein refers to linear or branched radicals. Examples of such radicals include, but are not limited to, methyl, ethyl, n-propyl, isopropyl, n-butyl, isobutyl, sec-butyl, tert-butyl, pentyl, isoamyl, hexyl and the like.

The term “alkenyl” as used herein refers to linear or branched radicals having at least one carbon-carbon double bond in a radical. Examples of alkenyl radicals include, but are not limited to, ethenyl, propenyl, allyl, propenyl, butenyl and 4-methylbutenyl. The term “alkenyl” include radicals having “cis” and “trans” orientations, or alternatively, “E” and “Z” orientations.

The term “alkynyl” as used herein refers to linear or branched radicals. Examples of such radicals include, but are not limited to, propargyl, butynyl, and the like.

The term “cycloalkyl” as used herein refers to saturated carbocyclic radicals. Examples of such radicals include cyclopropyl, cyclobutyl, cyclopentyl and cyclohexyl. The term “cycloalkyl” additionally encompasses spiro systems wherein the cycloalkyl ring has a carbon ring atom in common with the seven-membered heterocyclic ring of the benzothiepene.

The term “cycloalkenyl” as used herein refers to unsaturated carbocyclic radicals having at least one double bond. Cycloalkenyl radicals that are partially unsaturated carbocyclic radicals that contain two double bonds (that may or may not be conjugated) can be called “cycloalkyldienyl”.

The term “aryl” as used herein refers to a carbocyclic aromatic system containing one or more rings wherein such rings may be attached together in a pendent manner or may be fused. The term “aryl” includes, but is not limited to, aromatic radicals such as cyclopentodienyl phenyl, naphthyl, tetrahydronaphthyl, indanyl, biphenyl, and anthracenyl.

The term “heteroaryl” as used herein refers to fused or unfused radicals, preferably 3-10 membered fused or unfused radicals. Preferred examples of heteroaryl radicals include benzofuryl, 2,3-dihydrobenzofuryl, benzothienyl, indolyl, dihydroindolyl, chromanyl, benzopyran, thiochromanyl, benzothiopyran, benzodioxolyl, benzodioxanyl, pyridyl, thienyl, thiazolyl, furyl, and pyrazinyl. More preferred heteroaryl radicals are 5- or 6-membered heteroaryl, containing one or two heteroatoms selected from sulfur, nitrogen and oxygen such as thienyl, furanyl, thiazolyl, imidazolyl, pyrazolyl, isoxazolyl, isothiazolyl, pyridyl, piperidinyl or pyrazinyl. The term “heteroaryl” includes, but is not limited to, a fully unsaturated heterocyclyl. The term “heteroaryl” also includes all positional isomers. In the “heteroaryl” radical, the point of attachment to the molecule of interest can be at the heteroatom or elsewhere within the ring.

The expression “pharmaceutically acceptable carrier” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained.

The expression “effective amount” as used herein refers to a nontoxic but sufficient amount of the drug or agent to provide the desired effect.

According to one embodiment, the compound can be a compound of formula (I) and R₁ can be —OH. R₁ can alternatively be a phenyl.

According to another embodiment, the compound can be a compound of formula (II) and R₁ can be —OH. R₁ can alternatively be a phenyl.

The process of the present invention, can be carried out by reacting together the compound of formula (XV) (or metaboric acid) and glycerol and then heating, for example at a temperature of about 100° C. to about 180° C. The process can also be carried out by a) reacting together the compound of formula (XV) and glycerol at a temperature of about 100° C. to about 105° C.; and b) heating the mixture obtained in step (a) at a temperature of about 110° C. to about 180° C. Alternatively, the process can be carried out by: a) heating glycerol at a temperature of about 100° C. to about 105° C. and adding thereto the compound of formula (XV) so as to obtain a mixture; and b) heating the mixture at a temperature of about 110° C. to about 180° C.

The compositions of the present invention can be preferably in a form suitable for administration as following: topical, oral, rectal, inhalation, injection and absorption through mucous membrane. Such a form can be of a cream, a lotion, a solution, a gel, a paste, an emulsion, a foam, an inhalant, an aerosol, a liniment, a syrup, a transdermal patch (electrical or not), a soft melt tablet, a powder, an injectable (subcutaneous, intravenous, intramuscular, intraperitoneal, intracranial and intrathecal) a transdermal infusion, an implant, a liposomal system and a suppository. A cream is particularly preferred.

The compositions can comprise several other ingredients such as petrolatum, lanolin, polyethylene glycols, bee wax, mineral oil, diluents such as water and alcohol, and emulsifiers and stabilizers. Thickening agents may also be present.

Suitable thickeners include those conventionally employed in topical creams such as, for example, monoglycerides and fatty alcohols, fatty acid esters of alcohols having from about 3 to about 16 carbon atoms. Examples of suitable monoglycerides are glyceryl monostearate and glyceryl monopalmitate. Examples of fatty alcohols are cetyl alcohol and stearyl alcohol. Examples of suitable esters are myristyl stearate and cetyl stearate. The monoglyceride also functions as an auxiliary emulsifier. Other emollients or oleaginous material which may be employed include petrolatum, glyceryl monooleate, myristyl alcohol and isopropyl palmitate.

In the compositions of the invention, several other components can be used such as (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) benzyl alcohol as solvent (22) EDTA (ethylene diamine tetracetic acid) as chelating agent; (23) other non-toxic compatible substances employed in pharmaceutical formulations; and (24) water.

Suitable thickeners can also be used in the compositions of the present invention such as those conventionally employed in topical creams. Examples include but are not limited to beeswax, hard paraffin, monoglycerides and fatty alcohols, fatty acid esters of alcohols having from about 3 to about 16 carbon atoms. Examples of suitable monoglycerides are glyceryl monostearate and glyceryl monopalmitate. Examples of fatty alcohols are cetyl alcohol and stearyl alcohol. Examples of suitable esters are myristyl stearate and cetyl stearate. The monoglyceride also functions as an auxiliary emulsifier. Other emollients or oleaginous material which may be employed include petrolatum, glyceryl monooleate, myristyl alcohol and isopropyl palmitate.

The compositions of the present invention can also comprise triglyceride base such as triethanolamine or a preservative agent such as methylparaben(methyl-4-hydroxybenzoate), propylparaben(propyl-4-hydroxybenzoate) and phenetyl alcohol. They can also comprise a stabilizer such as ethylene glycol palmitostearate.

The compounds, compositions and methods of the present invention can be useful for treating various type of inflammation. As example, they can be used for treating articular inflammation, extra-articular inflammation, rheumatoid arthritis, juvenile chronic arthritis, skin inflammation, inflammation of soft tissues; inflammation presents in acute and chronic neurodegenerative disorders such as: stroke, Alzheimer disease, Parkinson disease, multiple sclerosis, amyotrophic lateral sclerosis (ALS or Lou Gehrig disease); AIDS dementia complex (HIV-associated dementia); inflammation presents in vascular disorders such as: myocardial infarction and essential hypertension (secondary to arterial stiffness); and inflammation with infection in cystic fibrosis (lower airway inflammation). They can alternatively be used for treating asthma, rheumatoid arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, acne, gingivitis, periodontitis, pruritus, decubitus ulcer, allergic dermatitis, seborrheic dermatitis, mummular dermatitis, actinic keratosis, actinic lentigos, ichthyoses, ichtyposiformis, Darier maladay, palmoplantary keratodermies, leucoplasiesl, eucoplasiformis, lichen, blister, collagen maladies, ultraviolet light damage, rosacea, melasma, comedons, polymorphs, conglobata, infection, sinusitis, dyshidrosis tonsillitis, bronchitis, buccal ulceration, laryngitis, orophoryngeal, mucositisrheumatoid spondylitis, osteoarthritis, gouty arthritis, uveitis, iritis, chronic rhinosinusitis, distal proctitis, gastrointestinal diseases, inflammatory bowel diseases, urogenital diseases, fungal, yeast, bacterial and viral dermatides and wounds.

The compounds and compositions of the present invention can be used for preparing medicament for treating medicament for treating inflammation, pain or musculo-skeletal disorders.

When at least one compound of the present invention is used for water treatment, the concentration of the at least one compound can be at least 10 mg/L at least 15 mg/L, at least 20 mg/L, at least 30 mg/L, at least 40 mg/L, at least 50 mg/L, at least 75 mg/L, at least 100 mg/L, or at least 200 mg/L.

When at least one composition of the present invention is used for water treatment, the concentration of the at least one composition can be at least 10 mg/L, at least 15 mg/L, at least 20 mg/L, at least 30 mg/L, at least 40 mg/L, at least 50 mg/L, at least 75 mg/L, at least 100 mg/L, or at least 200 mg/L.

For example, the water to be treated can be water of a pool, a spa or a hot tub. It can also be water of a fountain, agricultural water, home water, community water, or a reused water.

When treating water with at least one compound or composition of the present invention, the treatment can at least substantially prevent the presence of bacteria such as E. Coli fecal coliforms, P. Aeruginosa, S. Aureus, and mixtures thereof. The treatment can also at least substantially prevent the presence of algae. Moreover, the treatment can permit to obtain a low turbidity water. For example, the turbity thus obtained can be no more than about 0.6 NTU, no more than 0.5 NTU or no more than 0.4 NTU.

In accordance with another aspect of the present invention there is provided a process for preparing a compound of formula (XVI) and/or (XVII):

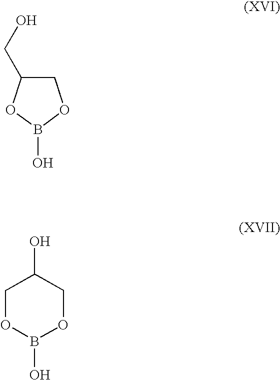

The process comprises the step of reacting together glycerol together with metaboric acid (HBO₂). Such a reaction is can be carried out at a temperature of about 100° C. to about 180° C.

### DETAILED DESCRIPTION OF PREFERRED EMBODIMENTS OF THE INVENTION

The present invention will be more readily understood by referring to the following examples which are given to illustrate the invention rather than to limit its scope.

In these examples, the following instruments and methods have been used. NMR spectra were recorded on a JEOL JNM-GSX270 FT NMR (1H 270 MHz; ¹¹B 87 MHz; ¹³C 68 MHz) spectrometer. Chemical shifts (δ) are reported in ppm [relative to residual solvent peaks (¹H and ¹³C) or external BF₃OEt₂ (¹¹B)] and coupling constants (J) in Hz. Multiplicities are reported as singlet (s), doublet (d), triplet (t), multiplet (m), broad (br), and overlapping (ov). Infrared spectra were obtained using a Mattson Genesis II FT-IR spectrometer and peaks are reported as broad (br), sharp (s) or weak (w). Melting points were determined using a MeI-Temp apparatus and are uncorrected. Elemental analysis was performed by Guelph Chemical Laboratories (Guelph, ON, Canada) under an atmosphere of dinitrogen. Mass spectroscopy was analysis was made using a Hybrid FT Mass Spectrometer>> from Thermo Electron corporation and having a resolution of 60000.

### Example 1

### Composition 1 Comprises Compounds 1 and 2

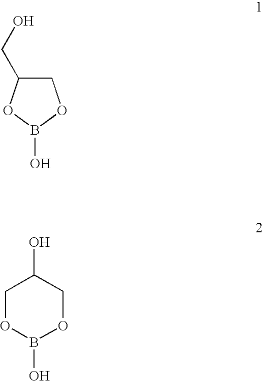

Composition 1 was prepared according to the general following procedure. In a first step, glycerol (OHCH₂CH(OH)CH₂OH) (53.0 g) was added to 100 ml 3-neck reaction flask. Temperature was raised to 92° C. and kept constant for 5 minutes. Boric acid (B(OH)₃) (40.0 g) was then added to the flask and a temperature of 100-105° C. was maintained for 30 minutes. In a second step, temperature was raised rapidly, over a period of less than 5 minutes, to about 120-125° C. and maintained for 130 minutes. Water generated during the reaction was vacuumed and transferred into a 20 ml volumetric separator, and weighted out on a balance (to 0.01 g). Approximately 25% of water (≈23 g) was collected at the end of this procedure. Finally, in a third step, temperature was increased rapidly (in less than 5 minutes) to 130-135° C. and more water was vacuumed, transferred and weighted out until an additional 5% of total initial weight was collected, over a 10-15 minutes time period. At this point, the maximum water collected from second and third steps preferably does not exceed 32%, so as to minimize the increase of viscosity of the mixture, thereby avoiding the product to have a semi-solid state.

The obtained Composition 1 was transparent with a faint yellowish color or colorless, depending on the source of reactants (glycerol, boric acid). It was then cooled to 90° C. and poured into glass containers. When temperature reached room temperature 20-24° C., containers were weighted and closed tightly since Composition 1 is hygroscopic. Yield of product was approximately 60%.

A detailed analysis of the above mentioned reaction was carried out was carried out. The reaction scheme was as follow:

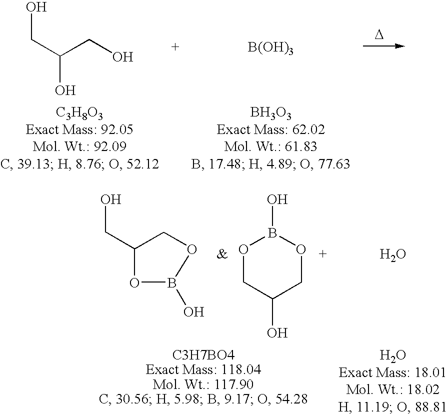

### Reaction Conditions

| | Glycerol + | Boric acid → | Product + | Water |
|---|---|---|---|---|
| wt | 53.12 | 40.05 | | 27.90 |
| MW | 92.05 | 62.02 | 118.04 | 18.01 |
| n | 0.577 | 0.646 | | 1.55 |

(wt = weight, MW = molecular weight and n = number of moles)

From the reaction conditions provided, it is clear that 2.6 equivalents of water are formed as a side product. While formation of the product borate esters (shown above) generate two equivalents of water, the addition 0.6 equivalents could arise from “wet” starting materials or further dehydration of the products to form a complex mixture of oligomers (diborate, triborate, & tetraborate esters, etc). However, elemental analysis is consistent with the monomeric products (Compound 1 and Compound 2), suggesting that the degree of oligomerization is minimal. The oligomers identified are as shown in formulas III to XIV.

It is interesting to note that the boron atom in the product mixture is always three coordinate, as demonstrated by ¹¹B NMR spectroscopy. Complexes containing four coordinate boron usually have peaks around 0 ppm. Two separate resonances are observed in solution and were assigned to borate ester derivatives containing a five membered ring (1,2-diol addition, Compound 1) and a six membered ring (1,3-diol addition, Compound 2). The nature of the products was also analyzed in various solvents using ¹H NMR and ¹³C NMR. While the ¹H NMR spectra shows a complicated overlapping of resonances, the number of peaks in the corresponding ¹³C NMR attests to the observation that there are more species in solution than the two simple isomeric products i.e. Compound 1 and Compound 2. Unfortunately, ¹³C NMR spectroscopy is usually not an accurate method of measuring relative intensities of products. Confirmation of multiple products is also observed in the various MS results. It is important to note that borates are remarkably sensitive to the nature of the MS experiment, and several different outcomes can be achieved depending upon the ionization conditions.

Presence of multiple oligomers in product was also confirmed by Mass Spectroscopy. It seems that we have primarily di, tri and tetraborate esters formation. Therefore, extend of oligomerization is relatively low and support a “loose” multibranching structural organization with the two isomers as initial building block elements. A high level of flexibility within that type of structure is thus expected, which should facilitate molecular-level, biological interactions in 3-D environment.

The composition obtained was stable under acidic, neutral and mild basic (pH≦8) conditions when tested as a 5% water solution. It was also noted that analogous complexes can be made using organoboronic acids, for example compounds of generic formula XV in which R₁ is different than —OH, instead of boric acid.

### Melting Point Determination

The initial melting point determination was performed in the air and due to the hygroscopic nature of Composition 1 an accurate melting point was not obtained.

Melting point range (air): 30-50° C.

By preparing and maintaining melting point samples under an atmosphere of dinitrogen, a more accurate melting point range was determined.

Melting point range (N₂) #1: 42-48° C.
Melting point range (N₂) #2: 42-49° C.
Melting point range (N₂) #3: 41-48° C.
Melting point range (N₂) average: 42-48° C.

### FT-Infrared Spectrum

The sample was prepared by suspending 30 mg of Composition 1 in Nujol under an atmosphere of dinitrogen. FT-IR (Nujol, cm⁻¹): 3647 (br, O—H), 2970 (s), 2947 (s), 2887 (s), 2841 (s), 1732 (w), 1653 (w), 1460 (s), 1377 (s), 1302 (w), 1153 (w), 1005 (w), 951 (w), 723 (w), 600 (w).

### Elemental Analysis

Approximately 20 mg of Composition 1 was sent to Guelph Chemical Laboratories. The sample was analyzed under dinitrogen and the composition was found to be: C, 31.15; H, 5.75.
1. The theoretical values for a 1:1 compound (glycerol:boric acid), C₃H₇BO₄, (117.90 gmol⁻¹) are: C, 30.56; H, 5.98.
2. The theoretical values for a 2:1 compound (glycerol:boric acid), C₆H₁₃BO₆, (192.00 gmol⁻¹) are: C, 37.53; H, 6.84.
3. The theoretical values for a 3:2 compound (glycerol:boric acid), C₉H₁₈B₂O₉, (291.89 gmol⁻¹) are: C, 37.03; H, 6.23.
4. The theoretical values for a 1:3 compound (glycerol:boric acid), C₃H₁₁B₃O₉, (223.57 gmol⁻¹) are: C, 16.12; H, 4.97.
5. The theoretical values for borospiranic acid, C₆H₁₂BO₄, (158.99 gmol⁻¹) are: C, 45.32; H, 7.62.

### Mass Spectroscopy

1. Instrument: LTQ—Orbitrap
[symbol] Hybrid FT Mass Spectrometer [symbol] from Thermo Electron corporation
Resolution: 60,000

2. Set up
Source: ESI+
Source voltage: 5.0 kv
Capillary voltage: 25.0 v
Capillary temp: 275° C.
Sheat gas flow: 9.0

3. Sample Injection
Volume 2.5 microliter
Mobile phase: 50% acetonitrile/0.2% formic acid/water

4. LTQ—Orbitrap Analysis: Composition 1
95% of the Orbitrap spectrum is composed of the following peaks:
193/210/215: —Correspond to 193 atomic unit (a.u.)
   Major peak (≈66% of all peaks)
   The ion m/z 210 correspond to ion 193+one ammonium
   ion (NH₄) from solvent impurities.
   The ion m/z 215 correspond to ion 193+one sodium atom.
   One boron atom per molecule: likely represents the two individual isomers.
293/310/315: —Two boron atoms per molecules
   Likely represents the dimers
   Minor peak: (≈24% of all peaks)
493/515: —Three boron atoms per molecule
   Likely represents the trimers
   Minor peak: (≈5% of all peaks)

### NMR Spectroscopy

NMR samples were prepared by dissolving 30-50 mg of Composition 1 in 1 mL of the appropriate solvent.

### 1. Composition 1 (MeOH-d₄, N₂, Wet Solvent)

¹H δ 3.89 (br m, minor peak), 3.69-3.59 (ov m), 3.57-3.47 (ov m).

¹³C{¹H} δ 72.5, 66.7, 64.7, 63.1.

¹¹B δ 17.6.

### 2. Glycerol (MeOH-d₄, N₂, Wet Solvent)

¹H δ 3.66-3.58 (ov m), 3.55-3.47 (ov m).

¹³C{¹H} δ 72.5, 63.1.

### 3. Boric Acid (MeOH, Air)

¹¹B δ 17.8.

### 4. Composition 1 (CDCl₃, N₂, Sparingly Soluble)

¹H δ 4.50 (m), 4.38 (m), 4.27-4.20 (ov m), 4.14-3.97 (ov m), 3.88 (m), 3.79 (m), 3.74 (m), 3.61 (m), 3.54 (m).

¹¹B δ 22.2, 17.2.

### 5. Glycerol (CDCl₃, N₂, Sparingly Soluble)

¹H δ 5.26 (br m), 5.13 (br m) 3.86-3.59 (ov m), 3.59 (br m), 3.46 (br m), 2.53 (d, J=4.7 Hz), 1.90 (t, J=5.7 Hz).

6. Boric acid (CHCl₃, Air)

¹¹B δ 17.9.

### 7. Composition 1 (CD₃CN, N₂)

¹H δ 5.91 (br s, 1H), 5.21 (br s, 1H), 4.32 (m, 2H), 4.11-3.77 (ov m, 15H), 3.49 (m, 1H).

¹³C{¹H} δ 76.5, 76.0, 75.6, 75.5, 75.4, 75.1, 68.3, 67.1, 66.9, 66.6, 66.4, 66.3, 66.1, 65.2, 64.6, 63.7, 63.6.

¹¹B δ 21.7, 17.0.

### 8. Glycerol (CD₃CN, N₂)

¹H δ 3.59-3.15 (ov m, 5H), 2.96 (d, J=4.7 Hz, 1H), 2.73 (t, J=5.7 Hz, 2H).

¹³C{¹H} δ 72.4, 63.3.

### 9. Boric Acid (CH₃CN, Air)

¹¹B δ 18.9.

### 10. Composition 1 (THF-d₈, N₂)

¹H δ 7.04 (m, 1H), 6.14 (m, 1H), 4.27 (m, 2H), 4.05-3.71 (ov m, 15H), 3.44 (m, 1H).

¹³C{¹H} δ 76.5, 76.0, 75.5, 75.4, 75.1, 75.0, 74.8, 74.7, 71.8, 70.9, 68.4, 67.2, 67.0, 66.6, 66.4, 66.3, 66.0, 65.5, 65.2, 65.1, 64.7, 63.9, 63.6.

¹¹B δ 22.2, 17.0.

### 11. Glycerol (THF-d₈, N₂)

¹H δ 3.83 (br m, 1H), 3.62 (br m, 2H), 3.55-3.37 (ov m, 5H).

¹³C{¹H} δ 72.7, 63.8.

### 12. Boric Acid (THF, Air)

¹¹B δ 19.1.

### 13. Composition 1 (Et₂O (Non-Deuterated), N₂, Sparingly Soluble)

¹¹B δ 22.5, 17.2.

### 14. Composition 1 (CH₃NO₂, N₂, Sparingly Soluble)

¹¹B δ 22.7, 17.9.

### Reactivity of Composition 1

### 1. Composition 1+xs LiOH in THF

Under an atmosphere of dinitrogen, LiOH (53 mg, 2.24 mmol) was added as a solid to a stirred THF (1 mL) solution of Composition 1 (50 mg, ˜0.2 mmol). The cloudy mixture was stirred at room temperature for 24 hours at which point the reaction was filtered and the filtrate analyzed by ¹¹B NMR spectroscopy.

¹¹B δ 21.7, 16.7.

### 2. Composition 1+xs LiOH in MeOH/H₂O

In the air, LiOH (53 mg, 2.24 mmol) was added as a solid to a stirred MeOH:H₂O (3:1 mL) solution of Composition 1 (50 mg, ˜0.2 mmol). The cloudy mixture was stirred at room temperature for 24 hours at which point the reaction was filtered and the filtrate analyzed by ¹¹B NMR spectroscopy.

¹¹B δ 9.0, 5.6, 1.9, 1.6, 0.6, 0.3.

### 3. Composition 1+3 Phenylboronic Acid in THF

Under an atmosphere of dinitrogen, phenylboronic acid (84 mg, 0.67 mmol) in THF (1 mL) was added to a stirred THF (1 mL) solution of COMPOSITION 1 (50 mg, ˜0.2 mmol). The clear solution was stirred at room temperature for 18 hours at which point the reaction was analyzed by ¹¹B NMR spectroscopy.

¹¹B δ 29.8 (phenylboronic acid), 24.8, 21.5, 17.9, 16.7.

### 4. Composition 1 in THF+Formic Acid

A 50 mg sample of Composition 1 was dissolved in 1 mL of anhydrous THF under an atmosphere of dinitrogen. The solution was removed from the dinitrogen atmosphere and 2 drops of formic acid were added to the solution while exposed to the air. The mixture was analyzed by ¹¹B NMR spectroscopy after 15 minutes and after 18 hours.

¹¹B (Composition 1, THF) δ 22.2, 17.0.

¹¹B (Composition 1+formic acid, 15 minutes, THF) δ 22.0, 17.2.

¹¹B (Composition 1+formic acid, 18 hours, THF) δ 22.0, 17.2.

### Control Reactions

### 1. Glycerol+B(OH)₃ in THF

Under an atmosphere of dinitrogen, B(OH)₃ (38 mg, 0.61 mmol) was added as a solid to a stirred THF (1 mL) solution of glycerol (50 mg, 0.61 mmol). The slighty cloudy mixture was stirred at room temperature for 24 hours at which point the reaction was filtered and the filtrate analyzed by ¹¹B NMR spectroscopy.

¹¹B δ 22.0, 19.1, 17.2.

### 2. Glycerol+3B(OH)₃ Heated in THF in the Presence of Molecular Sieves

Under an atmosphere of dinitrogen, B(OH)₃ (114 mg, 1.84 mmol) and activated molecular sieves (3 g) were added to a THF (5 mL) solution of glycerol (50 mg, 0.61 mmol). The reaction was heated to reflux for 4 hours at which point the reaction was filtered and the filtrate analyzed by ¹¹B NMR spectroscopy.

¹¹B δ 22.0, 18.9, 17.0.

### 3. Glycerol+Phenylboronic Acid in THF

Under an atmosphere of dinitrogen, a THF (1 mL) solution of phenylboronic acid (74 mg, 0.61 mmol) was added to a THF (1 mL) solution of glycerol (50 mg, 0.61 mmol). The pale yellow solution was stirred at room temperature for 24 hours at which point the reaction was analyzed by ¹¹B NMR spectroscopy.

¹¹B δ 30.3, 27.2, 25.5.

Under the conditions provided, it appears that addition of boric acid to glycerol generates a mixture of products derived from a double dehydration, whereby the resulting products contain either a five or six membered ring. The purity of the products can be assessed by a simple and inexpensive melting point determination (please note, the sample must be in a capillary tube free of water and dioxygen). It also appears that organoboronic acids can make similar complexes.

A stability test has been carried out on a batch of 1 Kg of Composition 1. The test was carried out over a period of 20 months. The long term stability of Composition 1 was observed over such a period of time. In fact, mass spectroscopy analyses confirmed that the product was stable, with the absence of degradation products and polymeric structures.

Composition 1 has also been prepared using another process. In fact, in this case metaboric acid (HBO₂) was used instead of boric acid. Metaboric acid and glycerol were thus reacted together as follows. In a first step, glycerol (OHCH₂CH(OH)CH₂OH) (23.2 g) was added to 50 mL 3-neck reaction flask. Temperature was raised to 100° C. and kept constant for 5 minutes. Metaboric acid (HBO₂) (17.5 g) was then added to the flask and a temperature of 105-110° C. was maintained for 15 minutes. In a second step, temperature was raised rapidly, over a period of 5 minutes, to about 135-140° C. and maintained for 60 minutes. Water generated during the reaction, was vacuumed and transferred into a 10 mL volumetric separator and weighted out on a balance (to 0.01 g). Approximately 15% of water (≈6.1 g) was collected at the end of this procedure. Finally, in a third step, temperature was increased over a period of 10 minutes to 170-180° C., and more water was vacuumed, transferred and weighted out until an additional 2% of total initial weight was collected over a 10-15 minutes time period. At this point, the maximum water collected from the second and third steps preferably does not exceed 18% so as to minimize the increase of viscosity of the mixture, thereby avoiding the product to have a semi-solid state.

The obtained Composition 1 was transparent with a faint yellowish color. It was then cooled to 90° C. and poured into glass containers. When temperature reached room temperature (about 20-24° C.), containers were weighted and closed tightly since Composition 1 was hygroscopic. Yield of product was approximately 75%.

### Example 2

### Compositions 2, 3 and 4

Several compositions were prepared by using, as active ingredient or anti-inflammatory agent, Composition 1. The general procedure for preparing Compositions 2 to 4 is the following. In each case three intermediate mixtures i.e. mixtures A, B and C have been prepared separately, and then these mixtures have been combined together so as to obtain Compositions 2 to 4. These compositions, which are in the forms of creams, are as follows.

### Composition 2

Mixture A:

| | |
|---|---|
| 1. Polyethylene glycol palmitostearate | 4.00% |
| 2. Capric triglyceride | 2.00% |
| 3. Cetyl alcohol | 4.20% |
| 4. Stearic acid | 6.00% |
| 5. Benzyl alcohol | 3.20% |
| 6. Propylene glycol stearate | 2.00% |
| 7. Beeswax white | 7.00% |
| Total: | 28.40% |

Mixture B:

| | |
|---|---|
| 1. Propylene glycol | 1.50% |
| 2. Methylparaben | 0.20% |
| 3. Propylparaben | 0.03% |
| Total: | 1.73% |

Mixture C:

| | |
|---|---|
| 1. Triethanolamine | 6.00% |
| 2. Sorbitol | 6.00% |
| 3. EDTA | 0.10% |
| 4. Composition 1 | 7.00% |
| 5. Water, Qs | 50.77% |
| Total: | 69.87% |
| Total: A + B + C | 100.00% |

### Composition 3

Mixture A:

| | |
|---|---|
| 1. Polyethylene glycol palmitostearate | 4.00% |
| 2. Capric triglyceride | 2.00% |
| 3. Cetyl alcohol | 3.50% |
| 4. Stearic acid | 7.00% |
| 5. Benzyl alcohol | 3.20% |
| 6. Polyethylene glycol (PEG) | 4.00% |
| 7. Phenetyl alcohol | 2.10% |
| 8. Sorbitan monostearate | 0.50% |
| 9. Beeswax white | 6.00% |
| Total: | 32.30% |

Mixture B:

| | |
|---|---|
| 1. Propylene glycol | 1.50% |
| 2. Methylparaben | 0.20% |
| 3. Propylparaben | 0.03% |
| Total: | 1.73% |

Mixture C:

| | |
|---|---|
| 1. Triethanolamine | 7.00% |
| 2. Sorbitol | 4.00% |
| 3. EDTA | 0.10% |
| 4. Composition 1 | 7.00% |
| 5. Water, Qs | 47.87% |
| Total: | 65.97% |
| Total: A + B + C | 100.00% |

### Composition 4

Mixture A:

| | |
|---|---|
| 1. Polyethylene glycol palmitostearate | 3.00% |
| 2. Capric triglyceride | 2.00% |
| 3. Cetyl alcohol | 3.50% |
| 4. Stearic acid | 6.00% |
| 5. Benzyl alcohol | 3.20% |
| 6. Polyethylene glycol (PEG) | 5.00% |
| 7. Sorbitan monostearate | 1.00% |
| 8. Beeswax white | 7.00% |
| Total: | 30.70% |

Mixture B:

| | |
|---|---|
| 1. Propylene glycol | 1.50% |
| 2. Methylparaben | 0.20% |
| 3. Propylparaben | 0.03% |
| Total: | 1.73% |

Mixture C:

| | |
|---|---|
| 1. Triethanolamine | 6.00% |
| 2. Sorbitol | 6.00% |
| 3. EDTA | 0.10% |
| 4. Diethyleneglycol monoethyl ether | 4.00% |
| 5. Composition 1 | 7.00% |
| 6. Water, Qs | 44.47% |
| Total: | 67.57% |
| Total: A + B + C | 100.00% |

### Example 3

### Tests on Various Subjects Using Composition 2

Composition 2 was tested on several subjects so as to verify its activity as anti-inflammatory agent and/or analgesic. As it can be seen from Table 1, test subjects having various inflammations or pain have been treated with Composition 2. In each case, a small amount of Composition 2, in the form of a cream, was applied to inflamed skin area by means of a short gentle rub. As demonstrated in Table 1, a complete disappearance of pain and/or inflammation in test subjects was observed.

TABLE 1
Use of Composition 2 for Treating Pain and/or Inflammation

| | | | | | | Pain Intensity (a) | | | | | |
|---|---|---|---|---|---|---|---|---|---|---|---|
| Volunteer/ | | | | Reason(s) for | | Before | After | Time to | Relapse | | Pain Condition |
| Patient | Age | Weight | | Inflammation | Localization | Appli- | Appli- | Pain Relief | of Pain | Number of | After Last |
| Code | (years) | (Kg) | Sex | & Pain | of Pain | cation | cation | (hrs) | (Y/N) | Applications | Application |
| D-123-S | 57 | 75 | F | Injury | Leg | ++ | + | 0.5 | Y | 3 | CD |
| D-633-J | 64 | 57 | F | Lower back pain, | Lower back, | ++ | 0 | 0.5 | N | 1 | CD* |
| | | | | Musculoskeletal | central | | | | | | |
| D-321-J | 39 | 55 | F | Fibromyalgia | Hands | +++ | 0 | 0.5 | N | 1 | CD* |
| D-457-D | 41 | 64 | F | Fibromyalgia | Hands & feet | ++ | 0 | 0.5 | N | 1 | CD* |
| D-942-L | 54 | 58 | F | Injury | Leg | +++ | ++ | 8.0 | Y | 3 | CD |
| D-701-M | 57 | 84 | M | Arthritis | Wrists | ++ | 0 | 0.5 | Y | 3 | CD |

All data were provided under testimony of all volunteers.
*The Composition 2 effect is higher than medications taken in the past to treat the same chronic afflictions (i.e.: acetaminophen/Tylenol ®, acetaminophen-codeine/Empracet ® or acetylsalicylic acid/Aspirin ®). Moreover Empracet ® and Aspirin ® were responsible for severe gastric burning sensation that prompted discontinuation of products.
The various terms and expressions used in Table 1 are as follows:
F: female
M: male
a) Pain intensity rating:
No pain: 0
Mild pain: +
Moderate pain: ++
Severe pain: +++ (i.e.: interference with sleep and/or day's activities).
b) Pain condition after the last application of Composition 2.
CD: complete disappearance of pain.
RP: residual pain (i.e.: equivalent to mild pain intensity).

Composition 2 reduced pain intensity for all volunteers. Four subjects out of six experienced a total disappearance of pain, 30 minutes after application of the cream containing Composition 1 i.e. Composition 2. There was a complete disappearance of pain for all subjects including those (three out of six) who required a maximum of three applications of Composition 2.

In view of the results presented in Table 1, it can be said that Composition 1, contained in Composition 2, is quite efficient as an analgesic and anti-inflammatory agent.

In fact, two of the subjects were afflicted with an injury to a leg and a complete disappearance of pain was observed within three days. Before the treatment, other subjects have been suffering from disease conditions such as lower back pain, fibromyalgia and arthritis in their chronic forms for at least three years. The pain was severe for one subject and moderate for three others with chronic disease condition. Following to the treatment using a cream containing Composition 1, for all subjects including the one with lower back pain, a complete relief of the pain was observed in approximately 30 minutes after the application. Moreover, only one out of four subjects had required additional applications (for a total of three) since pain relapsed approximately 12 hours later for the afflicted subject.

There was an evidence of inflammation for all four subjects with chronic state of disease; stiffness was common for all, whereas swelling at joints was present for subjects with fibromyalgia and arthritis. Another sign of inflammation, tenderness, was present on hands of one of the subject afflicted with fibromyalgia (subject code D-457-D). Following application of Composition 2, all four subjects with chronic diseases had a reduced sensation of stiffness within two hours and a complete loss of stiffness within four hours. Loss of swelling at joints and tenderness sensation disappeared in approximately the same time-frames as reported earlier for stiffness.

### Example 4

In order to evaluate Composition 1, a cytotoxicity assay was carried out and the results of this assay are as follows.

TABLE 2
In Vitro Cytotoxicity Evaluation of Composition 1 in
a Dose Range Assay with White Blood Cells (Monocytes)

| | Product Concentration | | Cell Count | % Inhibition |
|---|---|---|---|---|
| Product | μM | mg | (a) ×10⁶ | (b) |
| Composition 1 | 0 | 0 | 3.2860 | 0 |
| Composition 1 | 15.5 | 3.47 | 3.4450 | 0 |
| Composition 1 | 155.0 | 34.72 | 3.9235 | 0 |
| Composition 1 | 1550.0 | 347.20 | 1.8675 | 43.17 |
| Composition 1 | 15500.0 | 3472.00 | 1.8090 | 44.95 |

(a) Cells: Mononuclear white blood cells also called monocytes. Cell line is CBMC, for cord blood mononuclear cells. Cell counts were performed 7 days contact and compared with blank controls. Cell count value for each test represents an average of a duplicate cell counting.
(b) % Inhibition (on cell viability): Cell death was assessed microscopically by the technique of trypan blue dye exclusion.

As shown in Table 2, Composition 1 does not have any effect on cell viability at 155 μM, which represents approximately an in vivo intake of 2 grams. A 43% inhibition in cell number is obtained with 1550 μM; therefore indicating that Composition 1 is a safe product to utilize.

The above-mentioned results presented by way of examples only clearly demonstrate that the compounds of the present invention, and more particularly Composition 1, is very efficient as an anti-inflammatory agent or analgesic. It was also demonstrated that such compounds, that can be produced at low costs, can also be very useful for being used in the methods defined in the present invention. Moreover, it was shown that these compounds are safe.

### Use of Composition 1 for Wound Healing and Burns Treatment

Composition 1 fulfill many of the advantages that a boron derivative should have to work efficiently in wound healing. It was observed that Composition 1 is permits interconnection with the extracellular matrix at time of tissue repair. A solution of Composition 1 in glycerol (50%) was prepared and applied with a cotton swab over minor cuts to fingers of two subjects. Surprisingly, wound healing process was fast and scar-free. Another application of Composition 1 1 solution to a subject with deep lacerations on both lips (sport accident) approximately 1 cm long each resulted into a scar-free wound healing; no suture of damaged lips was necessary and no antiseptic were used. Composition 1 was applied once a day for three days. Composition 1 was also investigated for its ability to heal second degree burns. One subject was treated with astonishing success; there was instant relief to the pain that was associated with the burn (i.e.: accidental burn to the left hand by a hot plate), no blister formation and no scarring. Treatment was repeated for three days and traumatized skin was covered with sterile strips. There was no inflammation nor infection associated with the burn.

### Use of Composition 1 for Water Treatment

Composition 1 has been used for treating confined volumes of water. In a control above-ground swimming pool of approximately 45,000 liters, 1 kilogram (kg) of Compound 1 was dissolved in 20 liters of water (5% solution) and added directly to pool to achieve a final concentration of 22.2 mg/liter (or 22.2 ppm). Surprisingly the water became crystal clear seven (7) days later. Maintenance doses of Composition 1 (400 g) were added to swimming pool every period of 40 days as a single application. Regular microbiological and turbidity testing indicated an absence of bacteria (E. Coli fecal coliforms, P. Aeruginosa, S. Aureus) and a low water turbidity of ≦0.6 NTU (nephelometric turbidity units). Test period was 156 days (>5 months) with clear water. It is remarkable that no sanitizer such as chlorine and no algaecide nor oxidizer were used during the test period with Composition 1 in the pool. The method is particularly suited for treating recreational water such as swimming pools, spas and hot tubs. Composition 1 can also be used for other water treatment applications such as industrial and commercial (fountains) water treatment, agriculture, home water treatment, community water treatment, water re-utilization and others.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A compound of formula (I), (II), (III), (IV), (V), (VI), (VII), (VIII), (IX), (X), (XI), (XII), (XIII), or (XIV): 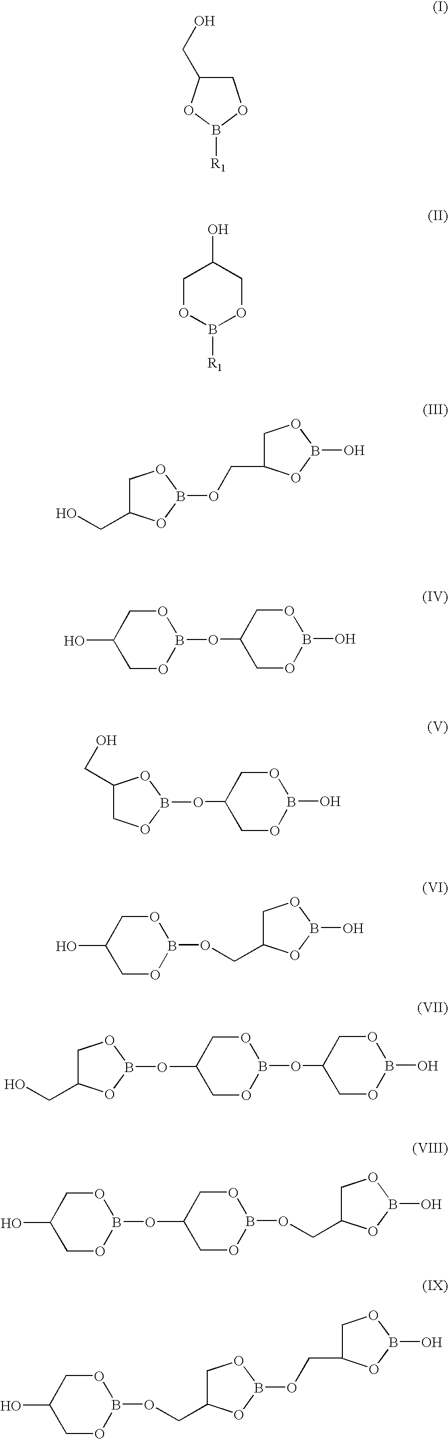 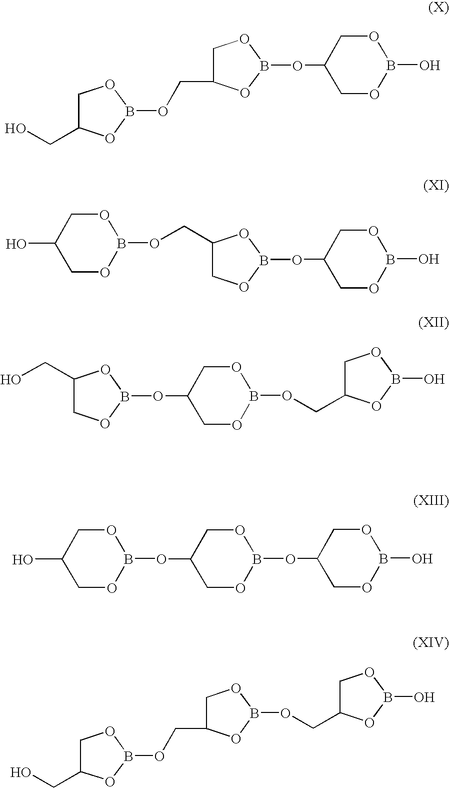
wherein
R₁ is —OH, a C₁-C₁₂ alkyl, a C₂-C₁₂ alkenyl, a C₂-C₁₂ alkynyl, a C₃-C₈ cycloalkyl, a C₃-C₈ cycloalkenyl, a C₆-C₁₂ aryl, or a C₁-C₁₂ heteroaryl group,
said alkyl, alkenyl, alkynyl, cycloalkyl, cycloalkenyl, aryl, and heteroaryl groups being unsubstituted or substituted by a hydroxy, a C₁-C₁₂ alkyl, a C₁-C₆ hydroxyalkyl, or a C₁-C₆ alkoxy.

2-7. (canceled)

8. The compound of claim 1, wherein said compound is a compound of formula (VI).

9. A composition comprising at least two compounds as defined in claim 1.

10-15. (canceled)

16. A composition comprising at least one compound chosen from compounds of formulas (I) and (II), and at least one compound chosen from compounds of formulas (III), (IV), (V), (VI), (VII), (VIII), (IX), (X), (XI), (XII), (XIII), and (XIV) 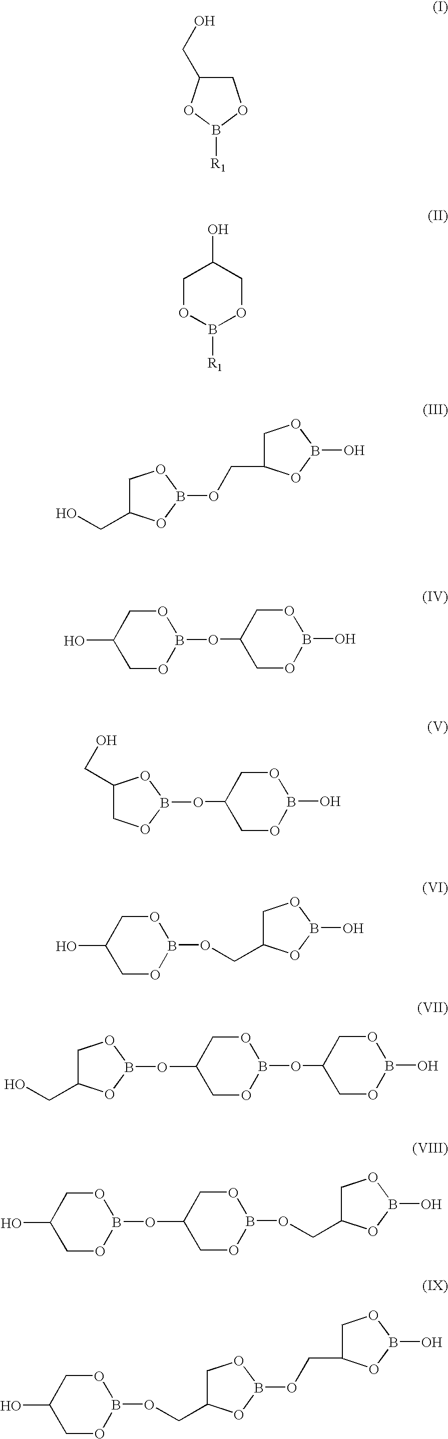 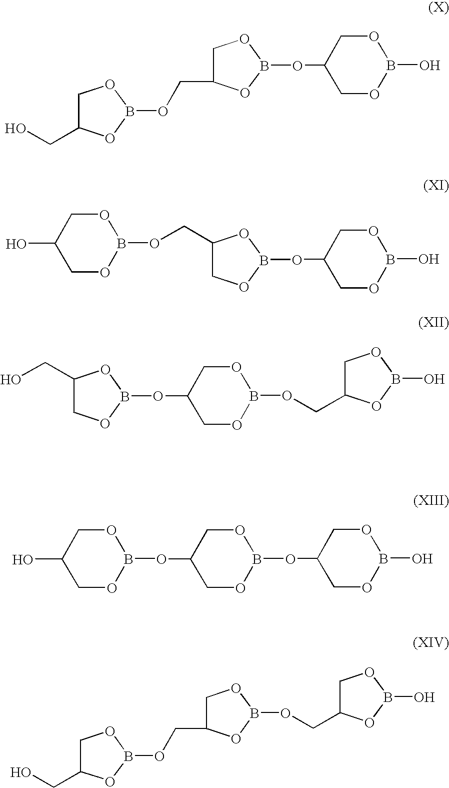
wherein
R₁ is —OH, a C₁-C₁₂ alkyl, a C₂-C₁₂ alkenyl, a C₂-C₁₂ alkynyl, a C₃-C₈ cycloalkyl, a C₃-C₈ cycloalkenyl, a C₆-C₁₂ aryl, or a C₁-C₁₂ heteroaryl group,
said alkyl, alkenyl, alkynyl, cycloalkyl, cycloalkenyl, aryl, and heteroaryl groups being unsubstituted or substituted by a hydroxy, a C₁-C₁₂ alkyl, a C₁-C₆ hydroxyalkyl, or a C₁-C₆ alkoxy.

17. The composition of claim 16, wherein said composition comprises a compound of formula (I) in which R₁ is —OH, a compound of formula (II) in which R₁ is —OH, and at least one compound chosen from compounds of formulas (III), (IV), (V), (VI), (VII), (VIII), (IX), (X), (XI), (XII), (XIII), and (XIV).

18. (canceled)

19. A kit for treating inflammation, pain or musculo-skeletal disorders comprising at least one composition as defined in claim 16, and instructions for using the same.

20. A method for treating at least one of inflammation and pain in a subject in need thereof, comprising administering to the subject an effective amount of at least one compound as defined in claim 1.

21. A method for treating at least one of inflammation and pain in a subject in need thereof, comprising administering to the subject an effective amount of at least one composition as defined in claim 16.

22-23. (canceled)

24. A method for treating musculo-skeletal disorders, comprising administering to a subject in need thereof an effective amount of at least one compound as defined in claim 1.

25. The method of claim 24, wherein said compound is topically administered to said subject.

26. A method for treating musculo-skeletal disorders, comprising administering to a subject in need thereof an effective amount of at least one composition as defined in claim 16.

27-36. (canceled)

37. A method for treating at least one of skin wounds and skin burns, comprising administering to a subject in need thereof an effective amount of at least one compound as defined in claim 1.

38. A method for treating at least one of skin wounds and skin burns, comprising administering to a subject in need thereof an effective amount of at least one composition as defined in claim 16.

39-40. (canceled)

41. A process for preparing a compound of formula (I) and/or (II): 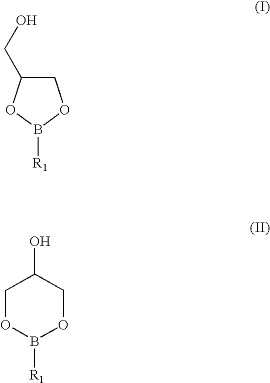
wherein
R₁ is —OH, a C₁-C₁₂ alkyl, a C₂-C₁₂ alkenyl, a C₂-C₁₂ alkynyl, a C₃-C₈ cycloalkyl, a C₃-C₈ cycloalkenyl, a C₆-C₁₂ aryl, or a C₁-C₁₂ heteroaryl group,
said alkyl, alkenyl, alkynyl, cycloalkyl, cycloalkenyl, aryl, and heteroaryl groups being unsubstituted or substituted by a hydroxy, a C₁-C₁₂ alkyl, a C₁-C₆ hydroxyalkyl, or a C₁-C₆ alkoxy,
said process comprising reacting a compound of formula (XV): 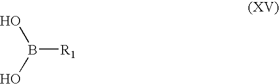
wherein R₁ is as previously defined,
with glycerol so as to obtain said compound of formula (I) and/or said compound of formula (II).

42. (canceled)

43. The process of claim 41, wherein said compound of formula (XV) and glycerol are reacted together by:
a) reacting together said compound of formula (XV) and glycerol at a temperature of about 100° C. to about 105° C.; and
b) heating the mixture obtained in step (a) at a temperature of about 110° C. to about 180° C.

44-49. (canceled)

50. A method for treating water, said method comprising contacting said water to be treated with at least one compound as defined in claim 1.

51. The method of claim 50, wherein said water is treated with said compound at a concentration of at least 10 mg/L.

52-53. (canceled)

54. A method for treating water, said method comprising contacting said water to be treated with a composition as defined in claim 16.

55-59. (canceled)

60. The method of claim 50, wherein said treatment at least substantially prevents the presence of bacteria.

61. (canceled)

62. The method of claim 50, wherein said treatment at least substantially prevents the presence of algae.

63-64. (canceled)
